SEQUENCE LISTING (2 sequences)

SEQ ID NO: 1 - protein, 51 aa, Artificial
  Insulin Oligomer
GIVEQCCTSICSLYQLENYCNFVNQHLCGSHLVEALYLVCGERGFFYTPKT

SEQ ID NO: 2 - protein, 51 aa, Artificial; identical to SEQ ID NO: 1
  Insulin Oligomer

(19) United States
(12) Patent Application Publication
(10) Pub. No.: US 2009/0099064 A1
(43) Pub. Date: Apr. 16, 2009

(54) Derivatized insulin oligomers

(75) Inventor: William D. Lougheed, Toronto (CA)

Correspondence Address: Gowling Lafleur Henderson LLP, Suite 1600, 1 First Canadian Place, 100 King Street West, Toronto, ON, M5X 1G5, CA

(73) Assignee: Diabecore Medical Inc.,, Toronto, ON (CA)

(21) Appl. No.: 11/808,299

(22) Filed: Jun. 8, 2007

Related U.S. Application Data
(60) Provisional application No. 60/811,766, filed on Jun. 8, 2006.

(51) Int. Cl. A61K 38/28; C07K 14/62

(52) U.S. Cl. 514/3; 530/303

(57) ABSTRACT

The present invention provides oligomers of phosphorylated insulin and formulations thereof. The oligomeric derivatives of the invention exhibit pharmacodynamic properties that are significantly improved over native insulin or other intermediate-acting or basal insulins, for example NPH, Lantus or Detemir, in that they demonstrate a 4-fold higher therapeutic index and a 4-fold lower risk of hypoglycemia. The invention provides the advantage of protracted glycemic lowering and combines it with the advantage of reduced hypoglycaemic risk. The above is not a property of any presently-known or available basal or intermediate-acting insulin. In a further embodiment of the invention, formulations of oligomeric phosphorylated insulin are suitable for all routes of administration including inhalation, buccal absorption, subcutaneous injection, infusion or other technically proven routes for insulin administration. The invention additionally provides the advantage of a longer-acting formulation for inhalation between meals and at bedtime. Such longer-acting inhalable formulations are not presently available.

### RELATED APPLICATIONS

This application claims the benefit of the priority filing date of U.S. Provisional Patent Application No. 60/811,766 filed on Jun. 8, 2006, which is incorporated herein in its entirety.

### BACKGROUND OF THE INVENTION

1. Field of the Invention

This invention is in the field of medicine and particularly the treatment of diabetes and hyperglycemia.

2. Description of Related Art

Diabetes mellitus is a debilitating disease that affects over 5% of the world's population. In the United States, approximately 15 million have diabetes. Symptoms of diabetes include hyperglycemia. The diabetic patient shows reduced production, release and/or sensitivity to insulin. Diabetes mellitus is classified as type I or insulin-dependent diabetes mellitus (IDDM) and type II or non-insulin-dependent diabetes mellitus (NIDDM). Type I diabetes, in which the pancreas has ceased producing insulin, affects 10% of all diabetics. It often begins in childhood and is known also as juvenile onset diabetes. In the more prevalent type II diabetes, or adult-onset diabetes, insulin production is maintained, but its' secretion in response to meals is delayed and/or diminished, and the diabetic's tissues are often less sensitive to insulin's effects.

In both types, diminished response to or low levels of insulin result in chronic high levels of blood glucose, which gradually alters normal body physiology and elevates the risk of micro-vascular and macro-vascular sequelae involving renal, cardio, retinal, neurological and circulatory complications. Such complications, arising principally from poor control of blood glucose, are a major health problem. Results from the Diabetes Control and Complications Trial (DCCT) demonstrate that improved glucose control lowers the incidence of disease complications [The DCCT Research Group, New. Engl. J. Med., 329, 977-986 (1993)].

However, achieving such control requires insulin therapy which mirrors the pattern of endogenous insulin secretion seen in normal individuals. In a healthy man, daily insulin secretion fluctuates according to need: (a) in the meal absorptive phase, insulin rises up to 10-fold to dispose of the meal-related glucose, amino acids & other fuels and (b) in the post-absorptive phase, a lower, relatively constant & sustained amount of insulin (basal supply) release occurs in order to balance hepatic (liver) glucose output with peripheral glucose uptake and thereby maintain normal blood glucose. Accordingly, effective therapy involves the combined use of two types of exogenous insulin: a fast-acting meal-time insulin and a longer-acting or intermediate-acting basal insulin.

HbA1C is an indicator of average prevailing blood glucose. The 2006 American Diabetes Association (ADA) treatment goals and recommendations include a glycosylated hemaglobin (HbA1C) as close to normal (<6%) as possible without producing significant hypoglycemia and a target morning pre-meal glucose of less than 130 mg/dL. Such ADA recommendations are based upon the observation that elevated glucose has a proven linkage with the disease's complications. In numerous large-scale multi-centre phase III clinical trials, to-date, no insulin or combination thereof has met the ADA treatment guidline for mean fasting blood glucose (FBG) of <130 mg/dL. Moreover, the usual best mean study population HbA1C's observed in large trials are not <6% but rather in the 7-8% range.

In phase 3 clinical trials, improved “basal” insulins, Lantus & Detemir have achieved FBG's of 145 mg %, still well above the ADA clinical recommendation and markedly above normal (<100 mg/dL). Unfortunately, existing insulins such as NPH or basal insulins such as Lantus or Detemir alone or in combination with fast-acting meal insulins such as LysPro fail to control glucose sufficiently well so as to meet existing medical treatment guidelines for fasting glucose on a day in day out basis. Clearly there remains great clinical need for improvement in basal insulin formulations that robustly control between-meal blood glucose.

As is well understood by those skilled in the art, long-acting insulin formulations have been obtained by formulating normal insulin as microcrystalline suspensions for subcutaneous injection. Examples of commercial basal insulin preparations include NPH (Neutral Protamine Hagedorn) insulin, protamine zinc insulin (PZI), and ultralente (UL). NPH insulin is the most widely-used insulin preparation, constituting until very recently from 50 to 70 percent of the insulin used worldwide. It is a suspension of a microcrystalline complex of insulin, zinc, protamine, and one or more phenolic preservatives. NPH-like preparations of a monomeric insulin analog, LysB298,ProB29-human insulin analog, are known in the art [De Felippis, M. R., U.S. Pat. No. 5,747,642, issued May 5, 1998].

Therapy using currently-available NPH insulin preparations fails to provide the ideal “flat” pharmacokinetics necessary to maintain optimal fasting blood glucose for an extended period of time between meals. Consequently, treatment with NPH insulin can result in undesirably high levels of insulin in the blood, which may cause life-threatening hypoglycemia.

In addition to failing to provide an ideal flat pharmacokinetics profile, the duration of action of NPH insulin also is inferior, often failing to last overnight. In particular, a major problem with NPH therapy is the “dawn phenomenon”, a hyperglycemia that results from the loss of effective glucose control before waking. Ultralente is crystalline preparation of insulin with higher levels of zinc than NPH, and not having either protamine or a phenolic preservative. Human ultralente preparations provide longer time action but the action is variable and inconsistent in effect.

Fatty acid insulins have been described [Whittingham, J. L., et al. [Biochemistry 36:2826-2831 (1997)] and insulin Detemir (Novo-Nordisk) has been recently released. The fatty acid chains used in fatty acid-acylated insulins are typically longer than about ten carbon atoms, and chain lengths of fourteen and sixteen carbon atoms are optimal for binding to serum albumin and extending time action. Fatty-acid acylated insulins such as Detemir are soluble at the usual therapeutic concentrations of insulin, but bind to albumin in the blood thereby providing prolonged action. However, the time action of these preparations is not sufficiently flat, to provide normal basal control.

Insulin Lantus is an insulin analogue with basal properties. It (as with Detemir) avoids some of the absorption variability observed with insulin suspensions by being insoluble at physiologic pH but soluble at the pH at which it is formulated. Upon injection it precipitation occurs and produces protracted absorption. Although reasonably flat absorption has been achieved with this insulin, the variability in day to day absorption combined with the steep dose-response curve of insulin does not allow sufficiently robust therapy to achieve ADA glucose targets without producing unacceptable hypoglycemia when this insulin was examined in large scale clinical trials.

Various phosphorylated insulins (P-Insulins) have been previously described. Insulin has been phosphorylated by methods employing phosphoric acid (Ferrel R. E. et al., Journal of the American Chemical Society, 70, 2107-7, 1948) or phosphoric acid/POCL.sub.3 in non-aqueous organic solvents using coupling agents (Cerami A. et al., U.S. Pat. Nos. 4,534,894 and 4,705,845) or with phosphoramidate (Rathlev, V. and Rosenberg T., Archives of Biochemistry and Biophysics, 65, 319-339, 1956). The phosphorylated insulin produced by Ferrel et el. and by Rathlev and Rosenberg were part of studies designed to further understand the process of phosphorylation and in particular to increase the knowledge of how it relates to biological systems. No clinical advantage of this phosphorylated insulin was measured or observed. None of the above prior art determined the specific amino acids or positions thereof on which insulin had been phosphorylated. Hence no specific molecular structure was disclosed or patented. Further, none of the above prior art discloses insulins with altered pharmacodynamics. In contrast, the present invention describes an insulin with a flattened glucose-insulin dose response curve.

The patents granted to Cerami et el. (cited above) involve the production of sulfated and phosphorylated insulin that have the advantage of reduced polymerization when stored long-term in insulin delivery systems. These insulins claim the advantage of not causing occlusion of insulin pumps and, accordingly, for those patients should produce better control of blood glucose. The Cerami at al. patents emphasize that the improvement in the process is attained by conducting the phosphorylation in a non-aqueous solvent. Accordingly, they teach of P-insulins with high ability to produce hypoglycemia (mouse convulsion assay). Thus, contrary to the results described here-in, the above Cerami et al insulins differ in that they do not exhibit reduced hypoglycaemic potential. Inter alia, one must conclude because of this, that the chemical identity of the P-insulins described by Cerami et differ markedly from the present invention. Cerami et al failed to determine the chemical identity of the heterogeneous mix (having a range of iso-electric pts) of phosphorylated insulins produced by their patents and chemical comparison by a person versed in the art is not possible without such.

Insulin has also been phosphorylated with POCl.sub.3 with excess pyridine as disclosed by Roubal Z et al., Chemical Abstracts, vol. 68, 1968, (Columbus, Ohio, U.S.). This reference discloses that insulin may be phosphorylated in anhydrous media with essentially no alteration of its hypoglycemic effect. The chemical composition is not disclosed and was not determined, nor was any beneficial effect in glucose control described or measured.

The author's patents (Lougheed, WD, U.S. Pat. No. 5,453,417 & PCT/CA92/00082) were allowed over the prior art cited here and over Albisser (U.S. Pat. No. 5,242,900). U.S. Pat. No. 5,242,900 by Albisser differs from the present invention in that it specifically describes monomeric P-insulins; “characteristically the modified product was intact monomeric insulin” (p. 4, 1.23-24). In contrast, the invention herein deals with insulin oligomers and the superior pharmacological action obtained with of oligomers. Albisser discloses amino acid sequencing results which are non-specific where one or more of three possible serine residues in insulin are phosphorylated as O-phosphates. No defined chemical composition of the phosphorylated insulin(s) can be made from this sequencing data.

The Lougheed patents (U.S. Pat. No. 5,453,417 & PCT/CA92/00082) describe a monomeric phosphorylated insulin having the unique property whereby insulin's steep dose response is flattened 3-4-fold with the proven advantage of reducing hypoglycemia. The reduce risk of hypoglycemia allows the insulin to be administered more aggressively with resultant improvement in controlling glucose. The present invention herein differs from the inventor's previous work in that it describes oligomers of phosphorylated insulin which have the surprising added utility of markedly prolonging its serum half-life. This property provides the extended action required for a basal insulin to control glucose between meals.

Pulmonary administration of insulin can be accomplished by either an aqueous solution or a powder preparation. Such dry powder, aerosol or liquid inhalable formulations of insulin have been extensively and successfully tested for example, U.S. Pat. No. 5,997,848, Patton, et al., Inhale Therapeutic Systems, Inc: Niven, Crit. Rev. Ther. Drug Carrier Sys, 12(2&3):151-231 (1995); Sayani et al., Crit. Rev. Ther. Drug Carrier Sys, 13(1&2): 85-184 (1996); U.S. Pat. No. 5,506,203; Platz et al., Inhale Therapeutic Systems, PCT WO 96/32149. Many have been shown to provide glucose control in Type I and Type II insulin-dependent diabetics for example; Patton, et al., Adv. Drug Delivery Reviews, 1, 35 (2-3), 235-247 (1999). The dry powder insulin formulations described by Patton, et al., overcome the problems of formulation instability.

Absorption may be increased by use of enhancers as reviewed by Sayani et al., Crit. Rev. Ther. Drug Carrier Sys, 13(1&2): 85-184 (1996) or by utilizing zinc-free formulations Boederke, P., U.S. Pat. No. 6,960,561. Dry powdered insulin may be formed into an aerosol by dispersing it into a gas stream, capturing it and meter dosing it as described by Patton et al., Inhale Therapeutic Systems, PCT WO 95/24183. Steiner S S, et al, U.S. Pat. No. 6,652,885 describe insulin-diketopiperazine precipitates. Such particles have a large fluted surface area which along with the removal of zinc enhances pulmonary absorption. Mannitol combined with various buffers may be used to increase stability of inhalable formulations as described by Platz et al., Inhale Therapeutic Systems, PCT WO 96/32149. Small particle sizes are optimum for deep lung penetration and are in the order 0.2-5 mu.m.

Long-acting pulmonary insulins have recently also been recently described in US patents: U.S. Pat. No. 6,465,426, Brader M L, issued Oct. 15, 2002; U.S. Pat. No. 6,838,076, Patton J S et al, issued Jan. 4, 2005; U.S. Pat. No. 6,310,038, Havelund S, issued Oct. 30, 2001; U.S. Pat. No. 6,335,316, Hughs B L et al, issued Jan. 1, 2002. These have the advantage of showing prolonged effect in pre-clinical studies for control of glucose between meals as tested in animal models. Efficacy in man has not yet been demonstrated. While these pulmonary formulations hold promise of providing basal pulmonary delivery, none demonstrate prolonged action combined with the advantage of reduced hypoglycaemic potential as is demonstrated in this application.

The American Diabetes Association professional advisory committee on diabetes has defined a need for extended-action insulins with reduced hypoglycaemic effect. [“more effort is needed to develop treatments which reduce the risk of hypoglycemia”, 1997 FDA Policy Statement on Diabetes].

The long serum half-life of oligomeric P-insulin compounds meet this need of providing long-acting formulations for treating diabetes while reducing the incidence of hypoglycemia. Therapy may be by any of the established treatment regimens, for example, by administration using needles, pens, inhalation systems, oral puffers, needle-less injection systems or insulin pumps.

### SUMMARY OF INVENTION

An improved insulin compositions which have both reduced hypoglycaemic effect and prolonged action for control of glucose between meals is provided. Surprisingly, the inventor has discovered that P-insulins of the sequence ID NO. 7 & 8 as defined below may be induced to form oligomers. The said oligomerization has the beneficial quality of:

1. prolonging absorption from the subcutaneous tissue and additionally extending the hormone's serum half-life. This has the effect, as compared to the inventor's previous patents, of thereby further reducing the hypoglycaemic risk; 2. remarkably extending the duration of action. Such pharmacodynamic properties enhance blood glucose control between meals.

The present invention thus relates to bulk drug substance and formulations of P-insulin oligomers in which such P-insulins importantly have the advantage of sustained duration of action and reduced hypoglycaemic effect. They are described by the oligomeric sequences of the form [X]ⁿ or more specifically by the sequences:

### X-X or

### X-X-X or

### X-X-X-X or

### X-X-X-X-X or

### X-X-X-X-X-X or

[X-]ₙ (wherein “X”- is repeated n times)
and wherein “X” is defined by sequence ID NO. 1 or ID NO. 2;

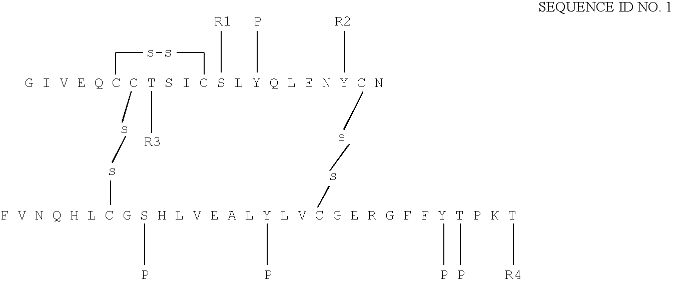

such that “—P” is defined as indicating an amino acid residue that is O-phosphorylated and such that “R” means an amino acid residue that may be additionally O-phosphorylated and wherein lower case “s” denotes sulphur;

### OR

Where “X” denotes sequence ID NO. 2;

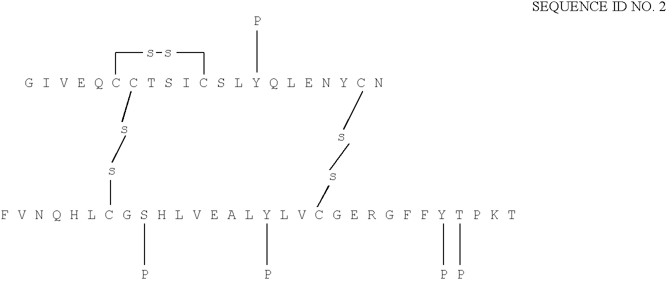

such that “—P” is defined as indicating an amino acid residue that is O-phosphorylated and wherein lower case “s” denotes sulphur.

### BRIEF DESCRIPTION OF THE DRAWINGS

The process of the invention will now be described with reference to the accompanying drawings, in which:

FIGS. 1A, B. Size Exclusion Chromatography on Q Sepharose in 99/1 50 mM Ammonium Phosphate, ph 9.0/acetonitrile. a. Oligomers (tetramers, hexamers and octamers). b. Molecular weight standards.

FIG. 1C. SDS polyacrylamide gel electrophoresis of phosphorylated insulin produced as per U.S. Pat. No. 5,453,417 showing only phosphorylated insulin monomer of molecular wt. 6,000 da.

FIG. 2A. Mass Spectroscopy of P-insulin oligomers. 2a. MALDI-MS of P-insulin oligomers. Fragmentation of higher oligomers into p-insulin monomer (5808 da), dimer (11,616) and trimer (17,424 da) occurs during MALDI-MS.

FIGS. 2B,C. P-insulin was reduced and alkylated to break the disulfide bond and separate the A and B chain. On MALDI-MS the masses of both A chain and B chain were observed and the results demonstrated 2 phospho sites in the A chain and 4 phospho sites in the B chain. MS/MS mapped the phosphorylation sites on the A Chain to Ser9 and Tyr16 (FIG. 2B). In order to map the remaining phosphorylation sites, the reduced and alkylated P-insulin was digested with trypsin overnight. The resulting fragments were shown in slides #15-17. Two phosphorylation sites were determined at Tyr26 and Thr27 of the insulin B Chain (FIG. 2C).

FIG. 3. IV injection of human insulin and P-insulin into pancreatectomized diabetic beagle dogs. A. human insulin showing rapid decline in blood glucose, hypoglycemia at 15 min. after injection and rapid rebound of glycemia due to short serum half-life. B. P-insulin oligomers at various doses. Note extended duration of action (long serum half-life), maintenance of normal glucose without hyperglycemia at all doses. Hypoglycemia was not observed until doses of 8 times the basal or “glucose maintaining dose” were reached.

FIG. 4. Subcutaneous injection of human insulin and P-insulin oligomers into diabetic beagle dogs. Note the extended duration of action as compared with unmodified insulin.

FIG. 5. Dose response curve of human & porcine & P-insulin oligomers in L6 rat myocytes. Cells were incubated for 60 min. in the various concentrations of each insulin in separate experiments. 2-deoxyglucose uptake was then measured. P-insulin olgomers not showed a flattened dose response at all concentrations investigated and surprising showed a maximal response that was markedly lower than that for porcine or human insulin.

FIG. 6. Glucose response of Sprague-Dawley rats to 40 mg of phosphorylated insulin hexamer. Rats were injected at Oh and glucose levels measured for 12 h. As shown, glucose levels dropped by 2 h post-dosing and remained suppressed out to 11 h post-dose, after which they returned to baseline or pre-dose levels.

This demonstrates the lessened hypoglycaemic potential of P-insulin oligomers even at overdose concentrations.

### DETAILED DESCRIPTION OF THE INVENTION

The following abbreviations are used to represent amino acids.

| Name | Abbreviation 1 | Abbreviation 2 |
|---|---|---|
| Alanine | ala | A |
| Arginine | arg | R |
| Asparagine | asn | N |
| Aspartic Acid | asp | D |
| Cysteine | cys | C |
| Glutamic Acid | glu | E |
| Glutamine | gln | Q |
| Glycine | gly | G |
| Histidine | his | H |
| Isoleucine | ile | I |
| Leucine | leu | L |
| Lysine | lys | K |
| Methionine | met | M |
| Phenylalanine | phe | F |
| Proline | pro | P |
| Serine | ser | S |
| Threonine | thr | T |
| Tryptophan | trp | W |
| Tyrosine | tyr | Y |
| Valine | val | V |

An insulin oligomer comprising one of the following sequences is provided:

X-X

X-X-X

X-X-X-X

X-X-X-X-X

X-X-X-X-X-X and,

[X-]ₙ X being repeated n times.

### X is any one of:

(a) an insulin defined by sequence ID NO. 1 or a pharmaceutically acceptable salt thereof: The sequence ID NO. 2 is:

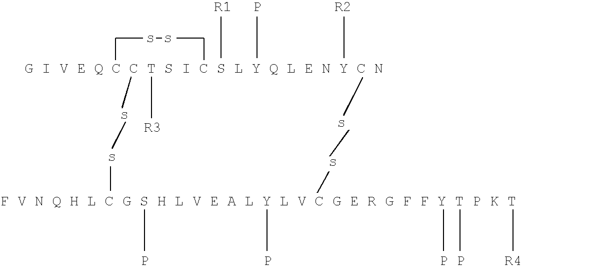

P indicates an amino acid residue that is O-phosphorylated,

R is an amino acid residue that is optionally O-phosphorylated, and lower case “s” is sulphur;

or,
(b) an insulin defined by sequence ID NO. 1 or a pharmaceutically acceptable salt thereof. The sequence ID NO. 2 is:

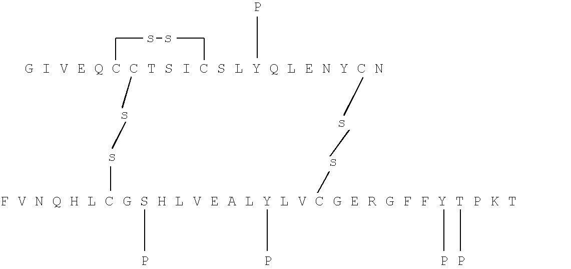

P″ indicates an amino acid residue that is O-phosphorylated, and

lower case “s” is sulphur.

The insulin defined by sequence ID Nos. 1 or 2 may be an insulin analogue.

In another embodiment, one or more of the residues at position R1, R2, R3 or R4 need not be phosphorylated.

A method for treating diabetes in a patient in need thereof is provided. The method comprises administering an effective dose of an insulin oligomer, as defined above, or analogues thereof.

Alternately, the method comprises administering by any one or a combination of subcutaneous infusion, inhalation, by buccal absorption, orally, or subcutaneous injection an effective dose of the insulin oligomer, or analogues thereof.

A method for treating hyperglycemia in a patient in need thereof is also provided. The method comprises administering orally an effective dose of an insulin oligomer, as defined above, or analogues thereof.

A composition containing insulin oligomer comprising one of the above sequences as defined above is also provided. The composition additionally comprises a pharmaceutically acceptable diluent, excipient or carrier therefor. The composition containing the insulin oligomer is used in the treatment of diabetes of hyperglycemia.

Use of an insulin defined by sequence ID NO. 1, sequence ID NO. 2 or pharmaceutically acceptable salts thereof, in the manufacture of an insulin oligomer comprising one of the sequences:

X-X

X-X-X

X-X-X-X

X-X-X-X-X

X-X-X-X-X-X and

[X-]ₙ X being repeated n times,

X being any one of the insulin of sequence ID. NO. 1 and sequence ID. NO. 2 is also provided.

Administration of phosphorylated insulin oligomers may be via any route known to be effective by the medical practitioner. Parenteral herein is defined as “administration by other than a gastrointestinal route”. Parenteral routes for administering the formulations of the present invention include subcutaneous, pulmonary, intraperitoneal, intraarterial, intramuscular, nasal, intravenous, and buccal routes.

The formulations of the present invention may make use of a catheter or an infusion system or a needle introduced into a catheter or under the skin or conversely may utilize an inhaler for pulmonary or buccal administration. Additionally, P-insulin oligomers provided by the present invention may be administered by any other means recognized in the art for parenteral administration. P-insulin oligomers of the present invention may equally be formulated as dry powders or as aerosols for absorption into the lung, nasal cavity, sublingual space or buccal mucosa.

The amount of P-insulin oligomer of the present invention that is administered to control glucose depends on a number of factors, among which are included, without limitation, the route of administration, the potency of the formulation, weight and age, type of diabetes, the route of administration and bioavailability by that route, the in-vivo half-life of the administered P-insulin, and the formulation. Bioavailability of insulin is known to be reduced via various routes including and particularly the nasal, buccal and pulmonary modalities and dosages need to be adjusted upward and titrated individually by physicians or other medical specialists trained in these routes of administration. Continuous administration can be accomplished by any physician skilled in the art of titrating insulin dosages. Intermittent administration can be similarly achieved by one skilled in the art and taking into consideration the dosages required to control in the interim period between dosing.

Insulin and P-insulins of the present invention can be made by any of a variety of recognized protein/peptide synthesis techniques. These include recombinant DNA methods, semi-synthetic methods, solid phase or solution methods and particularly methods known for incorporating amino acids or phospho-amino acids into peptides and for producing insulin analogues [R. E. Chance et al.: Diabetes Care 4, 147 (1982); Chance, et al., U.S. Pat. No. 5,514,646, issued May 7, 1996); “Current Methods of Phosphorylation of Biological Molecules”, Slotin L A, Synthesis: 737-752 (1977); EPO publication number 383,472, Feb. 7, 1996; Brange, J. J. V., et al. EPO publication number 214,826, Mar. 18, 1987]. These patents disclose the preparation of various peptides, amino acids, phosphorylated amino acid acids, insulin or insulin analogs with sufficient detail to enable one skilled in the art to prepare the P-insulin of in the present invention. Suitable kinases can be equally used to phosphorylate tyrosine, serine and threonine residues on insulin so as to produce products of the invention.

According to the present invention, p-insulin oligomers can be used in any of a variety of inhalation devices and prepared by methods known in the art for preparation of pulmonary insulin formulations as described in for example: Platz, et al., WIPO publication No. WO96/32149, published Oct. 17, 1996; Patton, et al., WIPO publication No. WO95/24183, published Sep. 14, 1995; U.S. Pat. No. 5,622,166, issued Apr. 22, 1997; Mecikalski, et al., U.S. Pat. No. 5,577,497, Nov. 26, 1996; Mecikalski, et al., U.S. Pat. No. 5,492,112, issued Feb. 20, 1996; Williams, et al., U.S. Pat. No. 5,327,883, issued Jul. 12, 1994; Goodman, et al., U.S. Pat. No. 5,404,871, issued Apr. 11, 1995; Rubsamen, et al., U.S. Pat. No. 5,672,581, issued Sep. 30, 1997; Gonda, et al., U.S. Pat. No. 5,743,250, issued Apr. 28, 1998; Rubsamen, U.S. Pat. No. 5,419,315, issued May 30, 199; Rubsamen, et al., U.S. Pat. No. 5,558,085, issued Sep. 24, 1996; Johnson, et al., U.S. Pat. No. 5,654,007, issued Aug. 5, 1997; Gonda, et al., WIPO publication No. WO98/33480, published Aug. 6, 1998; Rubsamen, U.S. Pat. No. 5,364,833, issued Nov. 15, 1994; Laube, et al., U.S. Pat. No. 5,320,094, issued Jun. 14, 1994; Eljamal, et al. U.S. Pat. No. 5,780,014, issued Jul. 14, 1998; Backstrom, et al., U.S. Pat. No. 5,658,878, issued Aug. 19, 1997; Backstrom, et al., 5,518,998, issued May 21, 1996; Backstrom, et al., 5,506,203, issued Apr. 9, 1996; Meezan, et al., U.S. Pat. No. 5,661,130, issued Aug. 26, 1997; Schultz, et al., U.S. Pat. No. 5,645,051, issued Jul. 8, 1997; Rubsamen, U.S. Pat. No. 5,364,838, issued Nov. 15, 1994; Rubsamen, U.S. Pat. No. 5,672,581, issued Sep. 30, 1997; Williams, U.S. Pat. No. 5,277,195, issued Jan. 11, 1994. The entire disclosure of each of the publications listed above is incorporated expressly herein by reference.

Aqueous formulations of the present invention may be prepared by dissolving P-insulins at pH 4.0-8.0. Although dissolution above pH 9.0 is possible, exposure must be brief or temperature reduced to limit de-amidation of insulin which is known to occur. Within the accepted pH range above, the dissolution and preparation may use any physiologically acceptable non-toxic salts, isotonicity agents and buffers, including without limitation, citrate, phosphate, acetate, sodium chloride, hydrochloric acid, phosphoric acid, sulphuric acid, ethyldiamine tetra acetic acid, tris-hydroxyaminomethane and glycerol. It is highly preferred that the formulation be made isotonic to humans, the method of doing so being obvious to one skilled in the art.

The nature and concentration of additives to formulations, order of addition of same, drug concentration, the pH, temperature during preparation may be optimized for the formulation and intended route of administration.

The term “P-insulin” means phosphorylated insulin oligomers comprising sequence ID NOs. 1 and 2, inclusive.

The term “phenolic preservative” means any of m-cresols, phenols, methylaparabens. A phenolic preservative is required in maintaining the sterility of insulin formulations of the present invention when these are used in vials, cartridges, injectors, or other devices where multiple use and/or multiple access to the formulation is mandated. Such is not a necessity for single use for example; cartridges, envelopes, dry powder packages as for example used in inhalation or aerosol devices.

The term “insulin analog” means proteins that have an A-chain and a B-chain that have substantially the same amino acid sequences as the A-chain and B-chain of human insulin, respectively, but differ from the A-chain and B-chain of human insulin by having one or more amino acid deletions, one or more amino acid replacements, and/or one or more amino acid additions that do not destroy the insulin activity of the insulin analog.

The term “treating” herein means the medical care of a patient having diabetes or hyperglycemia for which insulin administration is indicated for the purpose of lowering glucose and alleviating symptoms of hyperglycemia, and the metabolic sequelae resulting from such. “Treating” encompasses and means administering a dose of the present invention either in solid or liquid form to a diabetic patient for the purposes of restoring or maintaining euglycemia, preventing hyperglycemia, reducing hyperglycemia and to otherwise reduce, ameliorate or prevent the complications of diabetes.

The following examples are provided merely to further illustrate and describe the invention. The scope of the invention is not limited to the following examples.

### EXAMPLE 1

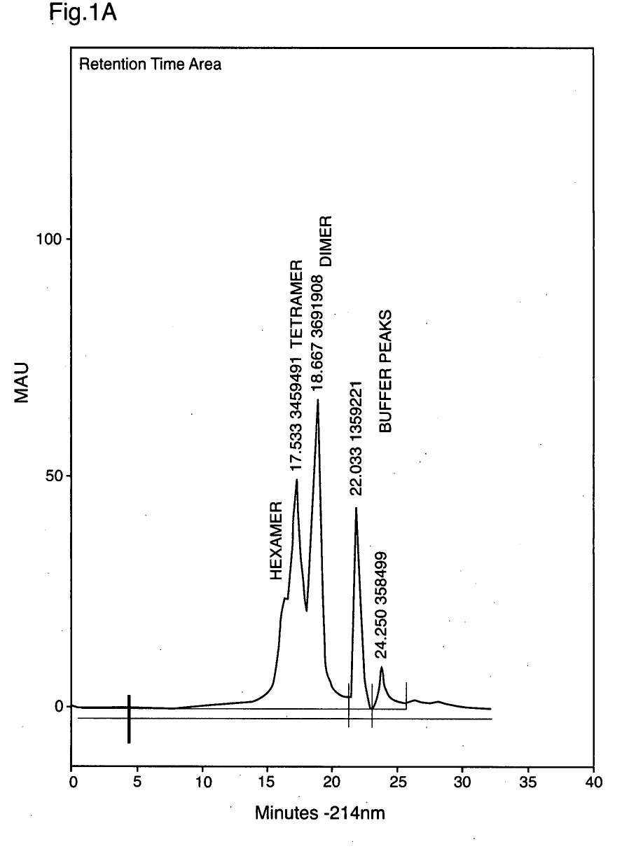

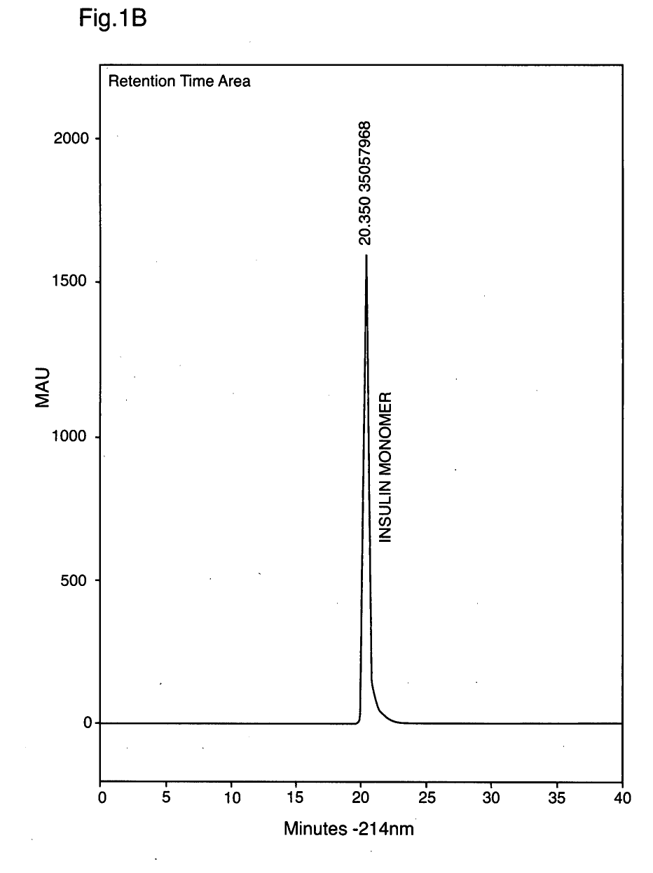

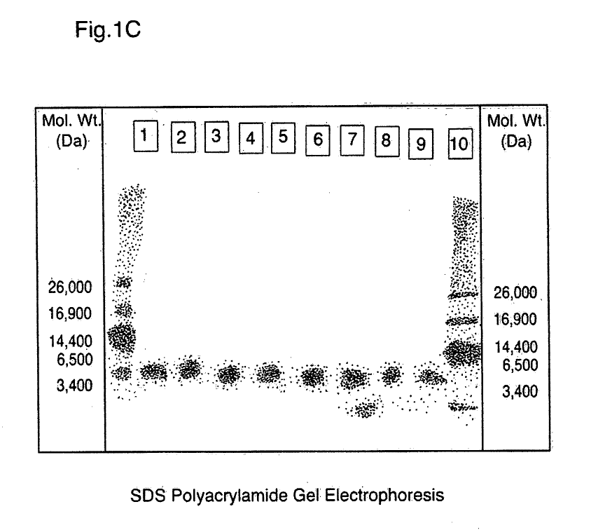

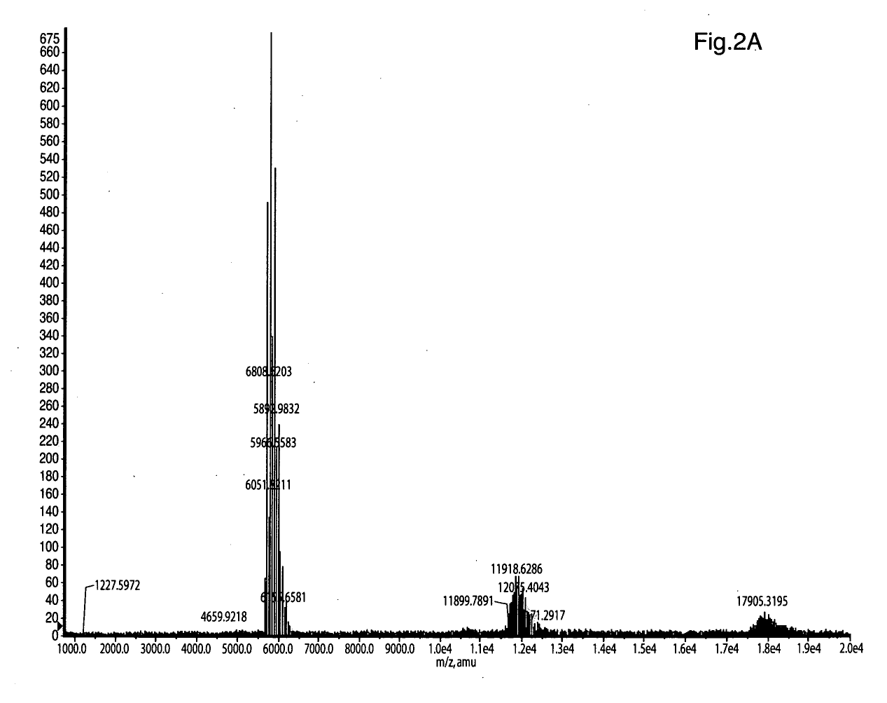

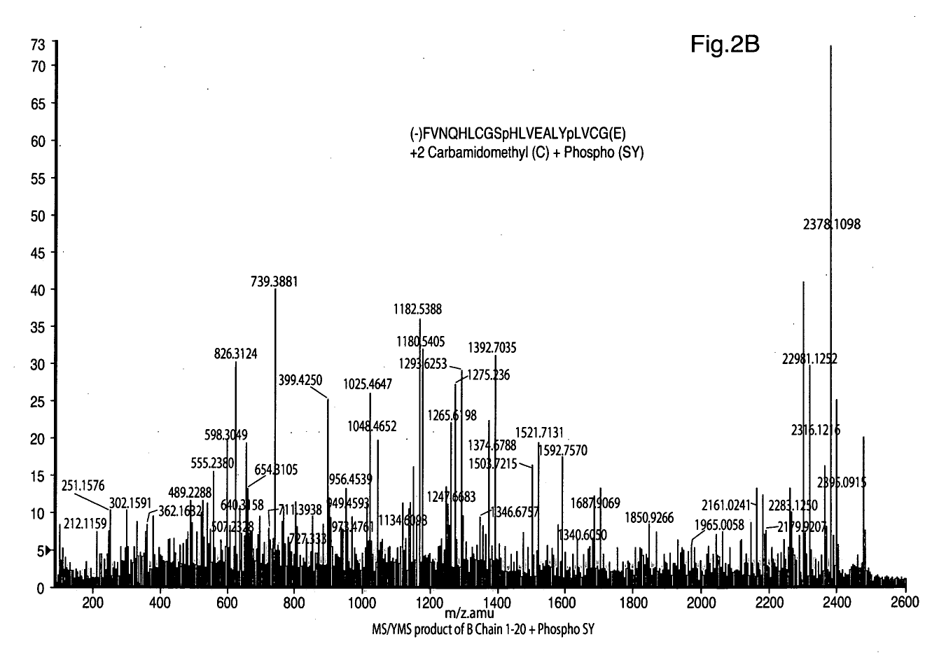

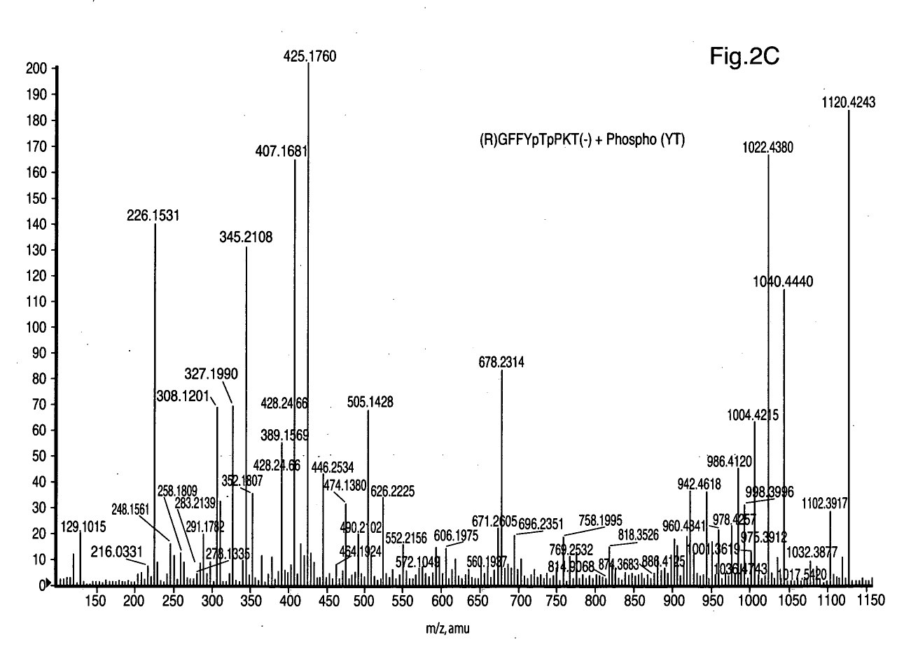

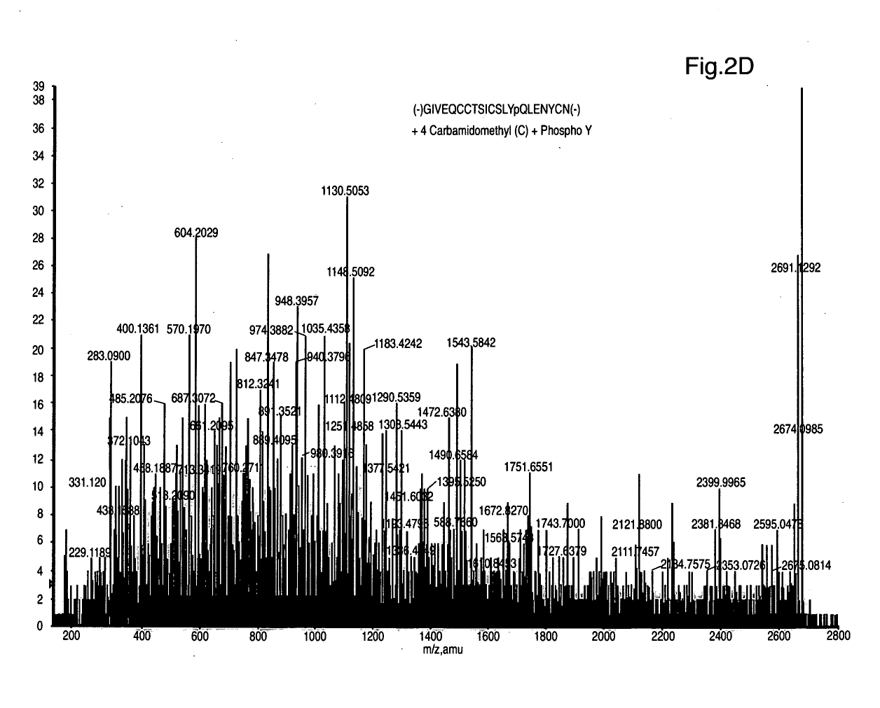

Insulin prepared by the methods described in (Markussen, et al, U.S. Pat. No. 4,916,212 filed May 29, 1985; issued Apr. 10, 1990) was dialyzed to remove all zinc, so as to optimise the post-reaction formation of appropriate monomeric precursors for P-insulin oligomers. Phosphorylation at −2 to +2° C. using a minimum starting concentration of 50 mM potassium phosphate, and excess POCl₃ (360 μL) for 60 minutes caused phosphate concentrations to reach solubility limits. This produced high yields of oligomeric phosphorylated insulin. Oligomers were separated by size exclusion chromatography on Zorbax 250 column, or similar columns using Q Sepharose, employing 50 mM ammonium phosphate buffer (pH 9.0) with 1% (v/v) acetonitrile. Dimers, trimers, tetramers and hexamers of phosphorylated insulin can be prepared in this manner as shown in FIGS. 1a, 2a. The retention times of the insulin monomer is shown in FIG. 1b for comparison. Samples were bracketed by HPLC analysis of appropriate molecular weight standards in order to determine mol. wts. The P-insulin monomer produced by U.S. Pat. No. 5,453,417 (Lougheed, W D) is shown for comparison in FIG. 1c. SDS polyacrylamide gel electrophoresis showed P-insulin monomer of MW 8,000 Da and the absence of insulin oligomers.

### EXAMPLE 2

Human Pro-insulin was prepared as per U.S. Pat. Nos. 4,559,300, Kovacevic, S. et al. issued Dec. 17, 1985, Di Marchi R., filed Aug. 1, 1983; 4,616,078, Di Marchi R, filed Oct. 7, 1986 and converted into human insulin by the methods described in U.S. Pat. No. 4,639,333, Obermeier R. et al., filed Jan. 27, 1987. Phosphorylation at −2 to 0° C. used a minimum starting concentration of 50 mM sodium phosphate, and excess POCl₃ (360 μL) for 60 minutes reaction. The reaction was quenched with 5-fold volume of de-ionized ice. High yields of oligomeric phosphorylated insulin were obtained. Oligomers were separated by size exclusion chromatography on a Zorbax 250 column, employing 50 mM ammonium phosphate buffer (pH 9.0) with 1% (v/v) acetonitrile. Dimers, trimers, tetramers and hexamers of phosphorylated insulin could be prepared in this manner

### EXAMPLE 3

A formulation of P-insulin oligomers was prepared as follows. 40 mg of P-insulin tetramer separated as above by size-exclusion chromatography was added to 9 mL of 15 mmolar sodium phosphate, 50 mmol NaCl. Glycerol was added to make the solution isotonic at the final volume. The pH was adjusted to 7.4 with 1 N NaOH and/or 1N HCl. M-cresol/phenol were added at equimolar concentrations of 0.125% (based on 10 mL final volume). Volume was made to 10 mL and the formulation sterile filtered and stored at 4° C.

### EXAMPLE 4

### Mass Spectroscopy

MALDI and Tandem Mass Spectroscopy were performed on P-insulin oligomers prepared in example 1, but maintaining the reaction temperature at 0° C. The results are shown in FIG. 2A-2D. As substantiated by iso-electric point determination using iso-electric focusing, the P-insulin monomer, dimer, trimer, tetramer and hexamer each contained 5 phosphate groups. Sulfotolysis to yield A&B chain and subsequent MALDI and Tandem mass-spectroscopy identified X in these oligomers as sequence SEQ. ID NO. 1 (FIGS. 2B-2D).

### EXAMPLE 5

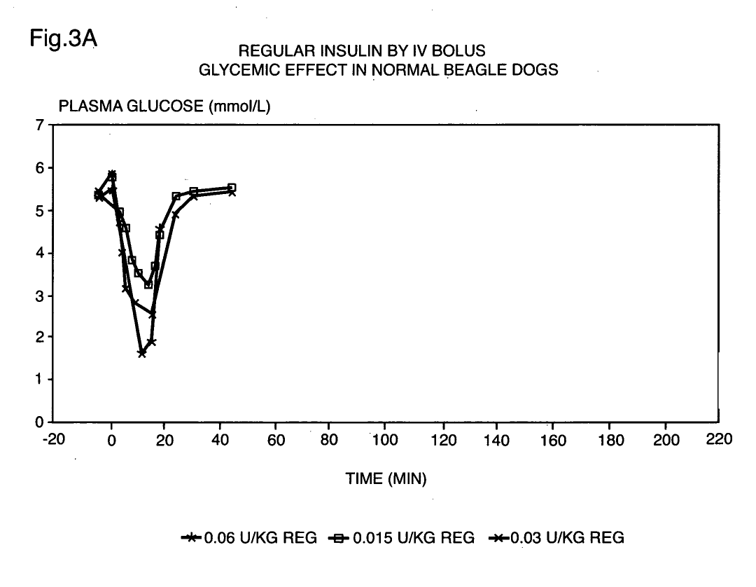

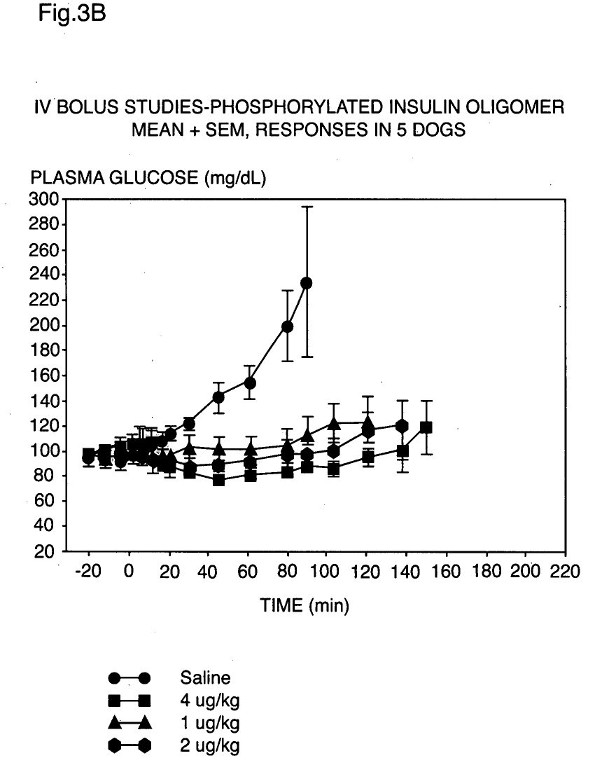

P-insulin oligomers of human insulin of sequence ID NO. 1 or unmodified human insulin were injected intraveneously into pancreatectomized diabetic dogs (FIG. 3). As shown in FIG. 3a unmodified insulin demonstrated a rapid decline in blood glucose, pronounced hypoglycemia and very short duration of action (20 minutes when given IV). In marked contrast oligomers of P-insulin FIG. 3b) showed minimal or no decline of blood glucose (except at very high doses), no hypoglycemia at all doses times and an up to 10 times longer action than unmodified insulin.

### EXAMPLE 6

P-insulin oligomers of human insulin of sequence ID NO.2 were injected subcutaneously into Fisher 344 rats (n=30). Injections up to 2,000 times the expected human dose induced hypoglycemia at only extremely high doses (approximately 0.15 mg/kg body wt.) and failed to induce convulsion or death.

### EXAMPLE 7

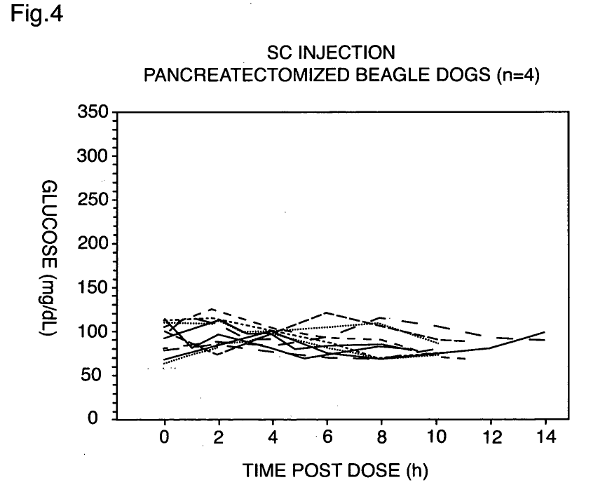

The formulation of example 3 containing P-insulin oligomers of sequence ID NO.2 and with 3:1 M:M protamine/insulin or unmodified insulin were injected subcutaneously into diabetic dogs. The prolonged glucose suppression of P-insulin oligomers is shown in FIG. 4.

### EXAMPLE 8

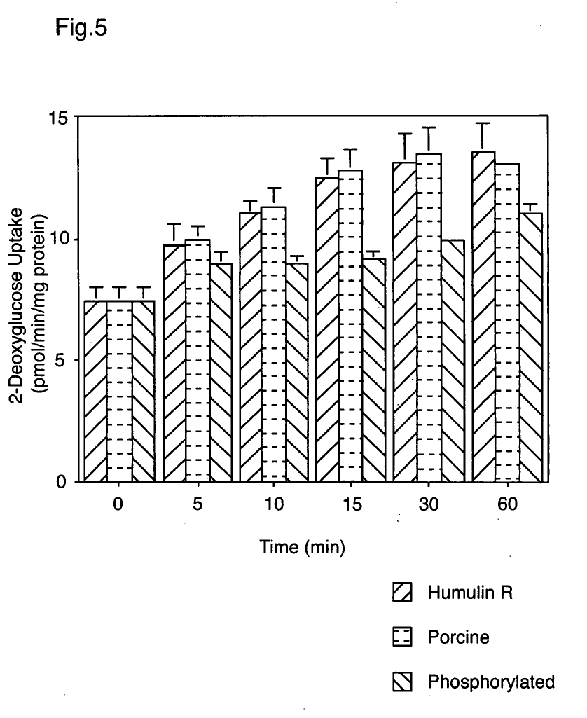

P-insulin oligomers of sequence ID NO. 1 were tested in L6 rat myocyte cell cultures for their ability to cause glucose uptake. P-insulin oligomers caused a maximal stimulation of deoxy-glucose uptake half that of human insulin and a flattened dose response over the entire dose range in rat muscle cells (FIG. 5).

### EXAMPLE 9

Via the pulmonary route, six Sprague-Dawley rats (BW 250-360 g.) were administered 4 mg of phosphorylated insulin oligomer (in the form of hexamer). The animals were dosed by intratracheal administration using a PennCentury intratracheal Aerosolizer (Microsprayer). The dose volume was 100 μL/animal. The dose concentration for the study was prepared by dilution of 30 mg/mL phosphorylated insulin hexamer in isotonic phosphate buffered normal saline, pH 7.4.

An Antisedan cocktail consisting of atipamezole hydrochloride (1 mg/mL, 1 mg/kg) and saline was administered by subcutaneous injection at a dose volume of 1 mL/kg, following completion of the dosing process. A series of 13 blood samples (approximately 0.1 mL/sample) were collected from each animal at 1, 2, 3, 4, 5, 6, 7, 8, 9, 10, 11 and 12 hours post-dose. For this purpose, each rat (unanesthetized) was bled by jugular/tail vein venipuncture (or other site as deemed necessary) and the sample used for measuring whole blood glucose levels using the Accu Soft Advantage™ (glucometer) method. Samples for plasma phosphorylated insulin levels were also taken.

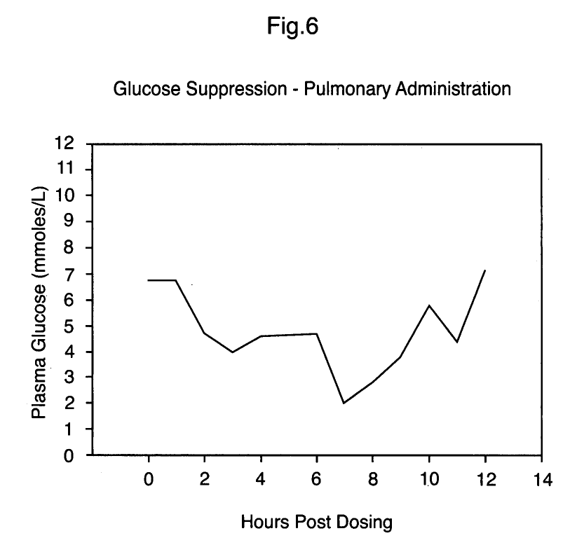

Following pulmonary administration (FIG. 6), blood glucose levels became moderately suppressed at 2 h and remained below baseline for 11 h post-dosing. By 12 h glucose levels returned to pre-dose levels.

It will be understood that other embodiments and examples of the invention will be readily apparent to a person skilled in the art, the scope and purview of the invention being defined in the appended claims.

I claim:

1. An insulin oligomer comprising one of the sequences:
X-X
X-X-X
X-X-X-X
X-X-X-X-X
X-X-X-X-X-X and,
[X-]ⁿ X being repeated n times;
X being any one of an insulin defined by sequence ID NO. 1 or a pharmaceutically acceptable salt thereof, the sequence ID NO. 1 being: 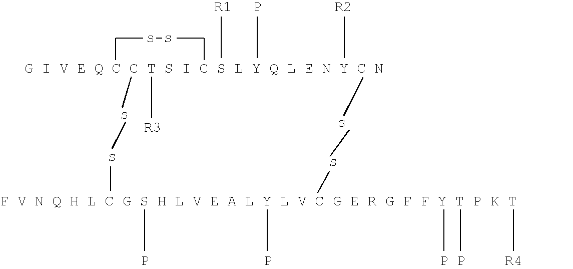
P indicating an amino acid residue that is O-phosphorylated,
R is an amino acid residue that is optionally O-phosphorylated, and
lower case “s” is sulphur;
or,
an insulin defined by sequence ID NO. 2 or a pharmaceutically acceptable salt thereof, the sequence ID NO. 2 being: 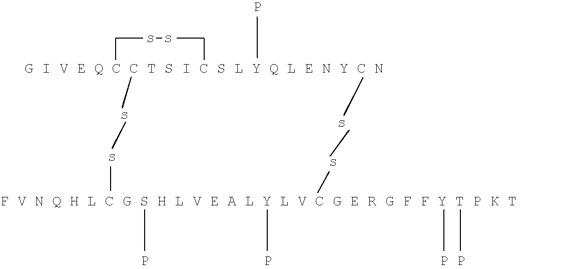
P″ indicating an amino acid residue that is O-phosphorylated, and
lower case “s” is sulphur.

2. An insulin oligomer of claim 1 where “X” is an insulin analogue.

3. An insulin oligomer of claim 1 wherein one or more of the residues at position R1, R2, R3 or R4 are not phosphorylated.

4. A method for treating diabetes in a patient in need thereof comprising administering an effective dose of an insulin oligomer of claim 1 or analogues thereof.

5. A method for treating diabetes according to claim 4 comprising administering by subcutaneous infusion or subcutaneous injection an effective dose of an insulin oligomer of claim 1 or analogues thereof.

6. A method for treating diabetes according to claim 4 comprising administering by inhalation an effective dose of an insulin oligomer of claim 1 or analogues thereof.

7. A method for treating diabetes according to claim 4 comprising administering by buccal absorption an effective dose of an insulin oligomer of claim 1 or analogues thereof.

8. A method for treating diabetes according to claim 4 comprising administering orally an effective dose of an insulin oligomer of claim 1 or analogues thereof.

9. A method for treating hyperglycemia in a patient in need thereof comprising administering orally an effective dose of an insulin oligomer of claim 1 or analogues thereof.

10. A composition containing insulin oligomer comprising one of the sequences:
X-X
X-X-X
X-X-X-X
X-X-X-X-X
X-X-X-X-X-X and,
[X-]ⁿ X being repeated n times;
X being any one of an insulin defined by sequence ID NO. 1 or a pharmaceutically acceptable salt thereof, the sequence ID NO. 1 being: 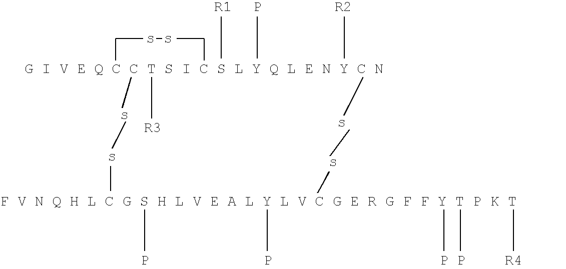
P indicating an amino acid residue that is O-phosphorylated,
R is an amino acid residue that is optionally O-phosphorylated, and
lower case “s” is sulphur;
or,
an insulin defined by sequence ID NO. 2 or a pharmaceutically acceptable salt thereof, the sequence ID NO. 2 being: 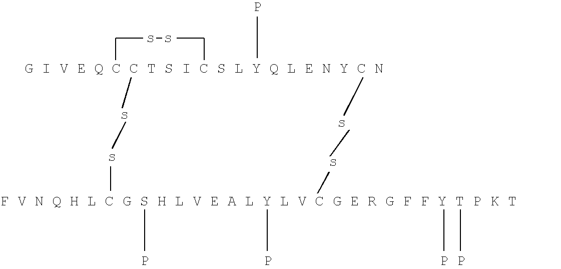
P″ indicating an amino acid residue that is O-phosphorylated, and
lower case “s” is sulphur,
and a pharmaceutically acceptable diluent, excipient or carrier.
